(19) United States
(12) Patent Application Publication
(10) Pub. No.: US 2026/0201384 A1
(43) Pub. Date: Jul. 16, 2026

(54) PHARMACEUTICAL COMPOSITION COMPRISING siRNA AND A METHOD OF PREPARING THE SAME

(71) Applicant: Reem MAHRAT, Pleasanton, CA (US)

(72) Inventors: Reem MAHRAT, Pleasanton, CA (US); Lonnie BOOKBINDER, Mesa, AZ (US)

(21) Appl. No.: 19/021,740

(22) Filed: Jan. 15, 2025

(51) Int. Cl. C12N 15/113; A61K 9/51; A61K 47/34; A61K 47/69

(52) U.S. Cl. CPC C12N 15/1136; A61K 9/5176; A61K 9/5192; A61K 47/34; A61K 47/6925; C12N 2310/14

(57) ABSTRACT

The present disclosure relates to a pharmaceutical composition comprising an inhibitory RNA molecule capable of targeting and inhibiting the expression of BLIMP 1 beta mRNA for the treatment of autoimmune disease. The present disclosure relates to combination therapy and mRNA inhibitory molecule that targets specific cells that causes autoimmune disease. The present disclosure also relates to method of preparing the pharmaceutical composition comprising siRNA, nanoparticle, cell targeting moiety, and PEG.

### REFERENCE TO SEQUENCE LISTING SUBMITTED ELECTRONICALLY VIA PATENT CENTER

The content of the electronically submitted sequence listing, file name: HuPRDM1bSiRNA.xml; size: 2.51 KB; and date of creation: Jun. 6, 2025, filed herewith, is incorporated herein by reference in its entirety.

### FIELD OF THE INVENTION

The present disclosure generally relates to methods and compositions for treating autoimmune diseases, and novel therapeutic approaches targeting specific immune pathways involved in these conditions. Particularly, the present disclosure relates to a pharmaceutical composition for treating autoimmune disease and a method of preparing the same. More particularly, the present disclosure relates to pharmaceutical composition comprising siRNAs that specifically targets the PRDM1B mRNA and the method of preparing the same.

### BACKGROUND OF THE INVENTION

Autoimmune diseases are a major healthcare concern worldwide. Autoimmune diseases are a cluster of clinically heterogeneous disorders characterized by chronic inflammation and multi-system involvements, which are caused by an immune-mediated attack on the body's own tissues and organs resulting from the dysregulation of immune systems to recognize self from non-self. The population affected by autoimmune diseases is estimated to reach at approximately 10% and is expected to keep increasing worldwide. Multiple sclerosis, type 1 diabetes, inflammatory bowel diseases, systemic lupus erythematosus, and rheumatic diseases are several examples. There are no cures for autoimmune diseases: Current therapies are anti-inflammatory drugs—to reduce inflammation and pain; corticosteroids—to reduce inflammation; pain medication and immunosuppressant drugs to inhibit the activity of the immune system. Small interfering RNAs represent a new and emerging therapy and have shown promising results in a number of diseases.

US20110150768 discloses the compositions and methods that make use of complexes comprising one or more inhibitory nucleic acids and a targeting polypeptide, wherein the targeting polypeptide consists of a cell surface receptor ligand. The compositions can be used in methods of silencing gene expression in a cell, in delivering agents to a target cell, and in treating or preventing a disease or disorder in a subject. This approach presents limitations.

The limitations of the approach disclosed in US20110150768, which utilizes inhibitory nucleic acids complexed with a targeting polypeptide (such as a cell surface receptor ligand). The limitations include lack of delivery efficiency as polypeptide-ligand complexes can be unstable in physiological conditions, which may reduce their ability to reach the intended cell or tissue. Further, there is limitation on their repeated or long-term use in therapy. The composition disclosed didn't have absolute specificity and have reduced their therapeutic efficacy due to degradation by nucleases. These compositions may face difficulties penetrating certain tissues or cells with barriers (e.g., blood-brain barrier), reducing their usability in specific disorders. Additionally, producing inhibitory nucleic acid-targeting polypeptide composition on a large scale is expensive and may not be easily scalable for widespread clinical use. In addition, the composition has disadvantages including transient effects, potential toxicity, receptor saturation etc.

Given the complexity of autoimmune diseases, there is an ongoing need for improved and precisely targeted therapeutics development. Therefore, there is a need for a composition that addresses the problem of inadequate treatment options for autoimmune diseases, which often involve complex immune-mediated responses and significant patient morbidity.

### OBJECTS OF THE INVENTION

An object of the present disclosure is to provide a pharmaceutical composition that addresses the disadvantages of the existing compositions.

Another object of the present disclosure is to provide a pharmaceutical composition that provides a new and effective approach to treat autoimmune disease.

Another object of the present disclosure is to provide a pharmaceutical composition for the targeted inhibition of PRDM1 protein expression through the use of inhibitory RNA molecules, designed to penetrate cell membranes and specifically bind to PRDM1 Beta mRNA.

Another object of the present disclosure is to provide a pharmaceutical composition that downregulates protein production associated with various autoimmune conditions, including multiple sclerosis, type diabetes, inflammatory bowel diseases, systemic lupus erythematosus, and rheumatic diseases.

Yet another object of the present disclosure is to provide a method of preparing the said pharmaceutical composition.

### SUMMARY OF THE INVENTION

The present disclosure relates to a pharmaceutical composition for treating autoimmune disease comprising: a) a BLIMP1 beta inhibitor having a double stranded siRNA molecule with 10-21 nucleotides complementary to human PRDM1 gene; (b) a pharmaceutically acceptable excipient; and (c) a drug delivery vehicle comprising a nanoparticle, wherein the double stranded siRNA has a sequence GTGTTACTTTAGGACTTGGA and GCAGAAATCAGGGCGGAAAC.

The present disclosure also relates to a method of preparing the pharmaceutical composition comprising the step of:
culturing and collecting cells from an exosome depleted media;
isolating exosomes from the cell culture media by differential centrifugation followed by ultracentrifugation to form pellets of exosomes;
purifying the formed exosomes; and
encapsulating doubled stranded siRNA into the exosomes.

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the scope of the present disclosures as defined by the appended claims.

The present disclosure provides solutions for the targeted inhibition of PRDM1 Beta protein expression through the use of inhibitory RNA molecules, designed to penetrate cell membranes and specifically bind to and inactivate PRDM1 Beta mRNA, corresponding to the mRNA of Blimp1 beta protein. Particularly, the present disclosure aims to downregulate protein production associated with various autoimmune conditions, including multiple sclerosis, type 1 diabetes, inflammatory bowel diseases, systemic lupus erythematosus, and rheumatic diseases, thereby addressing the need for more precise therapeutic interventions in the management of these disorders.

The term “siRNA” used in the present disclosure means small interfering RNA (siRNA). It is a synthetic, double-stranded RNA molecule that is used to silence genes and regulate gene expression. The siRNA is the double stranded siRNA with 10-21 nucleotides comprising a sense strand having the sequence GTGTTACTTTAGGACTTGGA and GCAGAAATCAGGGGGGAAAC. The double stranded siRNA molecule 4 is a member of sense strand 5, which contains double stranded siRNA molecule.

The term “siRNA” can be interchanged with the term “inhibitory RNA” in the present disclosure.

The term “BLIMP1 beta (β) inhibitor” used in the present disclosure comprises siRNA molecule with 10-21 nucleotides complementary to human Blimp1 beta mRNA. It is a transcriptional repressor that regulates immune cell development and function.

The term “PRDM1 gene” used in the present disclosure produces two isoforms of b lymphocyte-induced maturation protein 1 (BLIMP1 alpha and BLIMP1 beta). PRDM1 gene is a transcriptional regulator that plays a role in the development of many cell types. The PRDM1 gene produces mRNA of either the full-length protein BLIMP1 alpha or the truncated BLIMP1 beta isoform. The beta isoform is dysfunctional and does not perform all the activities of the alpha isoform, leading to a dysfunctional immune system unable to control inflammatory conditions, resulting in autoimmune diseases. Further, microbes, environmental chemicals and aging cause the PRDM1 gene to produce the dysfunctional PRDM1 protein Beta isoform and diminish the production of the PRDM1 alpha protein, leading to autoimmune diseases.

The present disclosure relates to a pharmaceutical composition comprising a BLIMP1 beta inhibitor, a pharmaceutically acceptable excipient, and a drug delivery vehicle.

In an embodiment of the present disclosure, the BLIMP1 beta inhibitor has a double stranded siRNA molecule with 10-21 nucleotides complementary to human Blimp1 beta mRNA. In yet another embodiment of the present disclosure, the double stranded siRNA has a sequence GTGTTACTTTAGGACTTGGA and GCAGAAATCAGGGCGGAAAC.

In another embodiment of the present disclosure, the composition further comprises polyethylene glycol (PEG). The PEG enhances the stability and solubility of the nanoparticle, and a cell targeting moiety that facilitates the selective delivery of the siRNA to target cells.

In yet another embodiment of the present disclosure, the double stranded siRNA molecule is encapsulated by the nanoparticle. The encapsulation of the BLIMP1 beta inhibitor by a nanoparticle complex enhances the stability and bioavailability of the inhibitor, allowing for more efficient delivery to the target cells. The nanoparticle complex facilitates targeted delivery of the BLIMP1 beta inhibitor, potentially increasing the concentration of the therapeutic agent at the site of inflammation while minimizing systemic exposure. The nanoparticle complex may allow for controlled release of the BLIMP1 beta inhibitor, providing sustained therapeutic effects and reducing the frequency of dosing required.

In yet another embodiment of the present disclosure, the composition also comprises a cell targeting moiety. A cell targeting moiety is a ligand that helps deliver molecules to specific cells. The cell targeting moiety is selected from the group consisting of an aptamer, antibody or part of an antibody or peptide.

In yet another embodiment of the present disclosure, the delivery vehicle is exosomes.

In yet another embodiment of the present disclosure, the pharmaceutical composition comprises the inhibitory RNA molecule in combination with a delivery system for targeted administration in autoimmune disease.

The pharmaceutical composition's delivery system facilitates targeted administration, potentially increasing the concentration of the inhibitory RNA molecule at the disease site and improving therapeutic outcomes. The combination with a delivery system may improve the stability and bioavailability of the inhibitory RNA molecule in the biological environment, extending its therapeutic window.

In another embodiment of the present disclosure, the pharmaceutical composition comprises double-stranded siRNA targeting PRDM1 encapsulated in exosomes. Particularly, the active ingredient of the present disclosure is double-stranded siRNA molecule. It is designed to target and modulate the expression of PRDM1 gene's expression of BLIMP-1 beta protein, a key, dysfunctional protein, a de-regulator of immune cell differentiation and function involved in autoimmune diseases. The siRNA is complementary to the mRNA encoding Blimp1 beta, promoting the degradation of Blimp1 beta and reducing Blimp1 beta protein levels.

The carrier is exosomes, which are naturally derived extracellular vesicles. These are used as nanocarriers for the delivery of siRNA to target cells (e.g., B cells, T cells, or specific tissues). These exosomes are isolated from donor cells, such as mesenchymal stem cells (MSCs) or immune cells, and are engineered to encapsulate the siRNA targeting Blimp1 beta. The diameter of the exosomes typically ranges between 30-150 nm. The exosomes are optionally engineered with targeting ligands on their surface to ensure selective delivery to specific immune cells (e.g., CD19 for B cells or CD3 for T cells).

In yet another embodiment of the present disclosure, the pharmaceutical composition comprises excipients and stabilizers. The excipients and stabilizers are selected from the group consisting of cryoprotectants, buffer solutions, and stabilizing agents. In another embodiments of the present disclosure, the cryoprotectants are agents like trehalose or sucrose to protect the exosomes and siRNA during storage and freeze-drying processes.

In another embodiment of the present disclosure, the buffer solution is a physiologically compatible buffer, such as phosphate-buffered saline (PBS), to maintain the stability of the exosomes and siRNA in the solution.

In yet another embodiments of the present disclosure, the stabilizing agents are the compounds such as polyethylene glycol (PEG) to prevent aggregation of exosomes and improve the formulation's shelf life and circulation time.

The pharmaceutical composition has exosome encapsulation. Particularly, the double-stranded siRNA targeting Blimp1 beta mRNA is encapsulated within exosomes, ensuring protection from enzymatic degradation (e.g., RNases) in the bloodstream. The exosomes naturally avoid detection by the immune system, enabling siRNA delivery with reduced immune activation or clearance.

Further, exosomes are functionalized with targeting ligands or antibodies that bind to specific surface markers on immune cells (e.g., B cells or T cells), allowing precise delivery of the siRNA to relevant cells involved in autoimmune diseases. By targeting Blimp1 beta mRNA with siRNA, the composition effectively reduces its expression, modulating the differentiation and function of immune cells like B cells and T cells. This modulation can reduce autoantibody production in diseases like lupus and rheumatoid arthritis, as well as suppress inappropriate T cell activity in diseases like multiple sclerosis.

The exosomes of the present disclosure are biocompatible, derived from natural sources, and present minimal toxicity or immunogenicity compared to synthetic nanoparticle carriers. They offer a safe and effective delivery system for siRNA therapies.

The pharmaceutical composition of the present disclosure may be administered through intravenous (IV) injection, which provides systemic distribution, allowing the exosomes to circulate and target relevant tissues or immune cells. The pharmaceutical composition may be administered through intraperitoneal (IP) or subcutaneous (SC) injection depending on the disease and desired localization of the therapeutic effect. Another administration may be local Injection for direct delivery to inflamed tissues or specific autoimmune target sites (e.g., inflamed joints in rheumatoid arthritis).

The pharmaceutical composition utilizing double-stranded siRNA targeting Blimp1 beta mRNA, encapsulated in exosomes, represents an innovative and targeted approach for treating autoimmune diseases. The encapsulation in exosomes enhances siRNA stability, ensures precise delivery, and mitigates off-target effects, making it a promising candidate for clinical application in precision medicine for immune modulation.

In yet another embodiment of the present disclosure, the pharmaceutical composition provides systemic delivery to a subject for the treatment of an autoimmune disease.

The present disclosure also relates to an inhibitory RNA molecule, also called as siRNA, that is capable of targeting and inhibiting the expression of Blimp1 beta mRNA for the treatment of autoimmune diseases. The siRNA molecule has 10-21 nucleotides complementary to human Blimp1 beta mRNA. It is double stranded and has a sequence GTGTTACTTTAGGACTTGGA and GCAGAAATCAGGGCGGAAAC.

In yet another embodiment of the present disclosure, the siRNA targets memory T regulatory cells that produces inflammatory cytokines in tissues leading to autoimmune diseases not only limited to type 1 diabetes, arthritis, colitis, Crohn's disease, multiple sclerosis and systemic lupus but also includes other autoimmune diseases. The BLIMP1 beta protein is responsible for such autoimmune disease. Therefore, the siRNA of the present disclosure blocks the mRNA of PRDM1 gene from producing the BLIMP1 beta protein. The inhibitory RNA molecules specifically target and inhibit the expression of PRDM1 BLIMP1 beta protein, providing a therapeutic effect in autoimmune diseases.

In yet another embodiment of the present disclosure, a therapeutic regimen for autoimmune diseases involves the use of the inhibitory RNA molecule as a targeted therapy to modulate immune response and mitigate inflammation. The inhibitory RNA molecules offer a novel and targeted approach for the treatment of autoimmune diseases, addressing the unmet need for precise and effective therapeutics in these complex conditions. The inhibitory RNA molecules provide a high degree of specificity in targeting the molecular pathways involved in autoimmune diseases, which may lead to fewer off-target effects and reduced side effects compared to conventional therapies. The targeted approach of inhibitory RNA molecules can result in a more potent therapeutic effect by directly modulating the immune response at the protein level, potentially leading to improved outcomes for patients. This therapeutic regimen can be tailored to individual patients based on their specific autoimmune disease and genetic makeup, offering a personalized medicine approach that could enhance the efficacy of treatment.

Further, the present disclosure relates to a method of inhibiting a protein that causes autoimmune diseases. The protein is an isoform of the PRDM1 gene and named as BLIMP1 beta. It is a truncated protein significantly different from the normal full-length protein BLIMP1 alpha. The alpha isoform regulates thousands of genes and proteins active in the immune system, whereas the beta isoform only regulates about half as many. The overexpression of the beta isoform leads to dysregulated T regulatory cells, which are normally immunosuppressive. As a result, the beta isoform causes autoimmune diseases and can be maintained over long periods of time.

The present disclosure also relates to a method for treating autoimmune diseases, comprising administering an effective amount of the inhibitory RNA molecule or the pharmaceutical composition to attenuate the immune-mediated responses associated with autoimmune conditions. Administering an effective amount of the inhibitory RNA molecule may lead to a reduction in the severity and progression of autoimmune diseases by attenuating immune-mediated responses. The said method offers a potential treatment option that can be tailored to the individual patient's needs based on the severity and type of autoimmune condition, allowing for personalized medicine approaches.

In yet another embodiment of the present disclosure, the autoimmune diseases include but are not limited to multiple sclerosis, type 1 diabetes, inflammatory bowel diseases, systemic lupus erythematosus, rheumatic diseases and all other autoimmune diseases. The broad applicability of the method of treatment indicates the potential for a versatile therapeutic strategy that can address various pathogenic mechanisms and benefits a wide patient population with different autoimmune disorders.

The present disclosure also relates to a method of preparing a pharmaceutical composition comprising the steps of: culturing and collecting cells from an exosome depleted media; isolating exosomes from the cell culture media by differential centrifugation followed by ultracentrifugation to form pellets of exosomes; purifying the formed exosomes; and encapsulating doubled stranded siRNA into the exosomes.

In an embodiment of the present disclosure, the isolation of the exosomes may be done using the differential centrifugation. The differential centrifugation includes low and high-speed centrifugation. The low-speed centrifugation is required to remove cells and large cell debris and high-speed centrifugation is required to remove larger vesicles and particles. Further, exosomes were ultracentrifuged to pellet exosomes. The other techniques to pellet exosomes include size-exclusion chromatography, density gradient centrifugation, or ultrafiltration may be used to isolate exosomes.

Further, the siRNA is encapsulated by the exosomes through electroporation process. Another technique for encapsulation includes incubation/Passive Loading. The direct incubating process include naturally incorporating siRNA by simple incubation. The exosomes and siRNA are mixed and incubated together at physiological temperatures. While, this method has lower loading efficiency compared to electroporation and might not ensure full encapsulation.

Another technique to encapsulate is transfection of donor cells. It involves transfecting the cells that produce exosomes with siRNA directly. Cells naturally package siRNA into exosomes during their biogenesis. After transfection, exosomes are collected from the transfected cells following the isolation steps outlined above. The siRNA will already be encapsulated within the exosomes. This method can yield exosomes pre-loaded with siRNA in a more natural and efficient manner without additional steps like electroporation.

Yet another approach is chemical Transfection (Lipofection). The Lipid-based transfection reagents (such as lipofectamine) can also be used to load siRNA into exosomes. The transfection reagent forms complexes with siRNA, facilitating its uptake into exosomes. This technique can enhance loading efficiency, but it may also affect exosome integrity and function.

Further, the inventors have carried out extensive research and experimentation to arrive at the present invention. They analyzed PRDM1 alpha protein (PR domain zinc finger protein 1), also known as BLIMP1, which is a crucial transcription factor that regulates the differentiation and function of B cells and T cells in the immune system. It is noted that dysregulation of PRDM1 alpha protein expression has been linked to several autoimmune diseases, where the immune system mistakenly attacks the body's own tissues. Therefore, it is found that modulating the expression of PRDM1 beta protein using siRNA (small interfering RNA) offers a promising therapeutic strategy to restore immune balance and control the overactive immune responses seen in these conditions.

Particularly, PRDM1 is a key regulator in the transition of B cells into plasma cells, which are responsible for producing antibodies. In autoimmune diseases, overactive plasma cells produce autoantibodies that attack healthy tissues. Modulating PRDM1 expression can reduce excessive plasma cell formation and decrease harmful autoantibody production. PRDM1 also plays an important role in controlling T cell activation, including the function of T regulatory cells (Tregs), which are involved in suppressing autoimmune responses. Therefore, proper modulation of PRDM1 expression can help rebalance T cell activity and limit the inflammatory response. Hence, the inventors have modulated PRDM1 expression by using siRNA. The siRNA works by inducing RNA interference (RNAi), where it binds to and degrades the messenger RNA (mRNA) of PRDM1 beta protein, preventing its translation into the BLIMP1 beta protein. By using siRNA to reduce or fine-tune PRDM1 induced protein expression of BLIMP1 beta protein, immune responses can be controlled more effectively, providing a means of treatment for several autoimmune diseases.

Further, the key mechanisms of siRNA modulation include partial Knockdown. It means instead of completely silencing Blimp1 beta mRNA, siRNA can be designed to partially reduce its expression, achieving a balanced modulation that reduces autoimmune activity without completely suppressing immune functions. Also, siRNA modulation targets gene silencing. Therefore, by selectively targeting Blimp1 beta mRNA in B cells or T cells, siRNA can modulate specific immune responses while leaving other critical functions intact. Moreover, modulating Blimp1 beta expression has therapeutic potential across several autoimmune diseases.

One of the autoimmune diseases is Systemic Lupus Erythematosus (SLE), which is characterized by the production of autoantibodies that attack various tissues, driven by excessive plasma cell activity. The siRNA modulation of Blimp1 beta mRNA downregulates Blimp1 beta mRNA expression and can reduce the differentiation of B cells into plasma cells, leading to decreased production of autoantibodies and alleviating disease symptoms.

Another autoimmune disease is Rheumatoid Arthritis (RA), wherein the chronic inflammation and joint destruction are driven by dysregulated B cells and T cells. Therefore, the siRNA modulation of PRDM1 can help reduce B cell differentiation into plasma cells and restore the regulatory function of T cells, ultimately reducing inflammation and slowing joint damage.

In case of Multiple Sclerosis (MS), it involves the immune-mediated destruction of the myelin sheath surrounding nerve cells, caused by autoreactive T cells. Hence, the siRNA modulation of PRDM1 can help rebalance immune responses and prevent the autoimmune attack on myelin, reducing the progression of MS.

In Type 1 diabetes, T cells attack the insulin-producing beta cells in the pancreas, leading to insulin deficiency. Thus, the siRNA modulation of PRDM1 can help reduce the activity of autoreactive T cells, preserving beta cells and potentially slowing the progression of the disease.

Having said the above, the benefits of modulating PRDM1 with siRNA includes selective immune modulation. The siRNA targets PRDM1 beta protein that allows for precise control over immune cell differentiation and function, reducing autoimmune activity without broadly suppressing the immune system. Further, it reduces autoantibody production by modulating PRDM1 beta protein expression in B cells. Thus, the siRNA can reduce the production of autoantibodies that drive tissue damage in diseases like lupus and rheumatoid arthritis. Another benefit includes control of inflammatory responses, wherein modulating PRDM1 in T cells can restore proper immune regulation, reducing the production of pro-inflammatory cytokines and alleviating chronic inflammation in autoimmune diseases.

The siRNA has the ability to fine-tune PRDM1 expression rather than completely silencing it. Therefore, it allows for more controlled therapeutic outcomes, minimizing the risk of immune suppression while addressing the overactive immune response. Further, effective delivery of siRNA to modulate PRDM1 expression is critical for its therapeutic success. Several strategies can be employed to deliver siRNA directly to target immune cells. The siRNA encapsulated in exosome offers a biocompatible and precise delivery system with low immunogenicity.

The invention is further illustrated by the following examples, which is provided to be exemplary of the invention and does not limit the scope of the invention. While the present disclosure has been described in terms of its specific embodiments, certain modifications and equivalents will be apparent to those skilled in the art and are intended within the scope of the present invention.

### EXAMPLES

The process for encapsulating double-stranded siRNA molecules in exosomes is as follows:

### Step 1: Isolating Exosomes

### A. Exosome Source

The cells were cultured cells that naturally secrete exosomes. The cells that were cultured from commonly used cells include mesenchymal stem cells (MSCs), dendritic cells, or other cell lines that produce large amounts of exosomes. Further, the cells were grown in exosome-depleted media to avoid contamination from exosomes naturally present in the media.

### B. Exosome Isolation

After the cells were grown, the culture medium was collected and subjected to differential centrifugation to remove larger particles and debris. The differential centrifugation includes low and high-speed centrifugation. The low-speed centrifugation is required to remove cells and large cell debris and high-speed centrifugation is required to remove larger vesicles and particles.

Further, exosomes were ultracentrifuged at 100,000×g in for 1-2 hours to pellet exosomes.

### C. Exosome Purification

Once the exosomes are isolated, it is further purified using exosome isolation kits, size-exclusion filters, or immunoaffinity capture techniques. This step ensures that only exosomes are present and removes contaminants or co-isolated vesicles.

2. Step 2: Process of Loading siRNA into Exosomes

After exosome isolation, the next step is to encapsulate the double-stranded siRNA within the exosomes. The siRNA is encapsulate using electroporation. For electroporation, exosomes and double-stranded siRNA are mixed in a solution. A brief electrical field is applied to create temporary pores in the exosome membrane. These pores allow the siRNA to enter the exosome. Once electroporation is done, the electrical field is stopped, and the exosome membrane resealed, trapping the siRNA inside. Further, the efficiency of siRNA encapsulation is improved by optimizing parameters like voltage, pulse duration, and siRNA concentration. Furthermore, the exosome-siRNA complex was purified to remove any free, unencapsulated siRNA.

3. Step 3: Characterization and Validation of siRNA-Loaded Exosomes

After loading the siRNA into exosomes, it's crucial to characterize and validate the encapsulation process to ensure efficiency, stability, and biological functionality.

Characterization of siRNA-Loaded Exosomes

| | Validation | |
|---|---|---|
| Parameter | Method | Passing Range/Criteria |
| Size (Exosome Diameter) | NTA/DLS | 30-150 nm (mean |
| | | size ~80-120 nm) |
| Polydispersity | DLS | <0.3 (indicates a homogenous |
| Index (PDI) | | population) |
| Concentration | NTA | ~10⁸-10¹² particles/mL |
| | | (depends on isolation) |
| Surface Markers | Western | Clear detection of ≥2 markers |
| (CD9, CD63, CD81) | Blot/ | |
| | ELISA | |
| Morphology | TEM | Intact, spherical vesicles with |
| | | smooth edges |
| Zeta Potential | DLS | ~−10 to −30 mV (slightly negative |
| | | charge) |

### A. Encapsulation Efficiency

Measured the amount of siRNA loaded into exosomes using methods such as quantitative PCR (qPCR) for detecting RNA. Then labeling the siRNA with fluorescent markers to quantify using fluorescence intensity. Further, siRNA concentration was measured using UV-vis spectroscopy.

### B. Exosome Size and Charge

The size distribution of the exosomes after siRNA loading were measured using Dynamic Light Scattering (DLS). Further, the surface charge of the exosomes were measured using Zeta potential to ensure that they maintain their stability and structure after siRNA loading.

C. siRNA Stability

The stability of siRNA inside exosomes were measured by checking for degradation using RNase treatment followed by quantification of the intact siRNA.

### D. Functional Validation

The functionality of siRNA-loaded exosomes were tested in cell cultures using in vitro assays to ensure that the siRNA successfully enters target cells and induces gene silencing. Further, the biodistribution, gene silencing efficiency, and therapeutic efficacy of the siRNA-loaded exosomes were evaluated in animal models using in vivo assays.

### 4. Targeted Delivery

To improve the specificity of siRNA delivery, exosomes were modified for targeted delivery. Exosomes were modified to carry specific ligands or antibodies on their surface, allowing them to target particular cell types or tissues. This was achieved through genetic engineering of the exosome-producing cells to express targeting peptides on exosomal surfaces. It was also achieved through chemical conjugation of targeting ligands to exosomal membrane proteins. Particularly, exosomes were engineered to express ligands that target specific cancer cells or tissues, improving the therapeutic potential of siRNA by focusing its effects on diseased areas while minimizing off-target activity.

| Parameter | Validation Method | Passing Range/Criteria |
|---|---|---|
| siRNA Loading | Fluorescence | 5-30% (loading efficiency |
| Efficiency | Spectroscopy/qPCR | typically observed) |
| Encapsulation | Quantification of | >80% siRNA encapsulated (low |
| Efficiency | unbound siRNA | free siRNA) |
| siRNA Integrity | qPCR/Gel | Intact siRNA bands observed |
| | Electrophoresis | |
| Exosome Yield | NTA/DLS | Minimal size/concentration |
| Post-Loading | | drop (<10%) |

### 5. Step 5: In Vitro and In Vivo Testing

Once siRNA-loaded exosomes were produced and characterized, they undergo rigorous testing. The in vitro testing is performed on cell culture to assess uptake of siRNA-loaded exosomes by target cells and efficacy of siRNA in silencing the intended target gene within the cells.

Further, in Vivo testing is conducted in animal models to determine biodistribution of the exosomes and to assess the therapeutic effect and gene silencing capability of the siRNA-loaded exosomes and to evaluate safety and potential immune responses.

### Cellular Uptake Assay

| | Validation | |
|---|---|---|
| Parameter | Method | Passing Range/Criteria |
| Uptake Efficiency | Flow Cytometry/ | >70% cells show fluorescent |
| | Confocal | siRNA signals |
| | Microscopy | |
| Colocalization | Confocal | >60-80% colocalization |
| (Exosome & siRNA) | Microscopy | within cells |
| Exosome Viability | MTT/Cell | ≥90% cell viability |
| | Viability | post-treatment |
| | Assay | |

### Functional Gene Silencing Validation

| | Validation | |
|---|---|---|
| Parameter | Method | Passing Range/Criteria |
| mRNA Knockdown (qRT- | qPCR | >50% reduction in target |
| PCR) | | gene expression |
| Protein Knockdown | Western Blot | >50% reduction in target |
| (Western Blot) | | protein levels |
| Control Gene Stability | qPCR | No significant changes |
| | | in control genes |
| Functional Assay (e.g., | Dual-Luciferase | ≥50% reduction in |
| Luciferase) | Reporter Assay | luciferase activity |

### In Vivo Biodistribution and Delivery

| Parameter | Validation Method | Passing Range/Criteria |
|---|---|---|
| Tissue-Specific | IVIS Imaging/qPCR | Signal enrichment in |
| Delivery | | target tissues ≥50% |
| siRNA Stability | qPCR/Biodistribution | Intact siRNA observed |
| (In Vivo) | | up to 48 hours |
| Systemic Toxicity | Serum Biochemistry | Within normal |
| | (ALT/AST) | physiological ranges |
| Organ Histology | H&E Staining | No significant tissue |
| | | damage observed |

### Notes for Validation Data Interpretation:

1. Loading Efficiency: Achieving ≥20% siRNA loading is ideal; however, ranges of 5-30% are acceptable, depending on the loading method.
2. Gene Knockdown: For effective silencing, both mRNA and protein levels should show a >50% reduction relative to untreated controls.
3. Exosome Integrity: Post-loading, size, concentration, and morphology should show minimal changes (<10%) compared to native exosomes.
4. Cellular Uptake: Confirm at least 70% internalization of siRNA-loaded exosomes in target cells for robust functional studies.
5. Toxicity: Exosome treatments should not reduce cell viability below 90% in vitro or show organ damage in vivo.
   Therapeutic Applications of siRNA-Encapsulated Exosomes

Exosomes encapsulating siRNA have promising therapeutic potential for treating a wide range of diseases, including:
Autoimmune Diseases: The composition is designed to treat autoimmune diseases such as Systemic Lupus Erythematosus (SLE), Rheumatoid Arthritis (RA), Multiple Sclerosis (MS), and Type 1 Diabetes by targeting and modulating the expression of PRDM1 in immune cells.
Chronic Inflammatory Conditions: In addition to autoimmune diseases, this composition may be applied in chronic inflammatory diseases where PRDM1 dysregulation is implicated.

### ADVANTAGES OF THE PRESENT DISCLOSURE

The inhibitory RNA molecule provides a targeted therapeutic approach, potentially reducing off-target effects and minimizing systemic toxicity compared to broader immunosuppressive treatments. By specifically inhibiting PRDM1 beta protein expression, the molecule offers a novel mechanism of action, potentially overcoming resistance to existing autoimmune disease therapies.

The present disclosure provides the advantage of enhanced immunity modulation, allowing for a more targeted approach in treating autoimmune diseases. The formulation offers the advantage of precise delivery and efficient action of the inhibitory RNA molecule at the cellular level. The composition provides the advantage of heterogeneity, enabling a versatile application various autoimmune conditions with differing pathophysiology. It also provides a tailored therapeutic approach that can be adapted to various conditions and patient responses. The therapeutic regimen provides the advantage of synergy, maximizing the therapeutic effects of the inhibitory RNA molecule in conjunction with the delivery system.

Furthermore, the present disclosure provides the advantage of isolation of the therapeutic mechanism, allowing for a clearer understanding of the treatment's effects and facilitating the development of personalized medicine strategies for autoimmune disease management. The siRNA penetrates cell membranes and specifically bind to PRDM1 mRNA to downregulate protein production. The cell membrane penetration capability of the molecule ensures efficient delivery to the intracellular environment where PRDM1 mRNA is located, enhancing the therapeutic efficacy. The specificity for PRDM1 mRNA is precise, potentially reducing the risk of unintended interference with other cellular processes.

### SPECIFIC EMBODIMENTS OF THE PRESENT DISCLOSURE

The present disclosure relates to a pharmaceutical composition for treating autoimmune disease comprising:
a) a BLIMP1 beta inhibitor having a double stranded siRNA molecule with 10-21 nucleotides complementary to human PRDM1 gene;
(b) a pharmaceutically acceptable excipient; and
(c) a drug delivery vehicle comprising a nanocarrier,
wherein the double stranded SiRNA has a sequence GTGTTACTTTAGGACTTGGA and GCAGAAATCAGGGGGGAAAC.

Such pharmaceutical composition is disclosed, further comprising a PEG.

Such pharmaceutical composition is disclosed, wherein the double stranded siRNA molecule is encapsulated by exosomes.

Such pharmaceutical composition is disclosed, further comprising a cell targeting moiety.

Such pharmaceutical composition is disclosed, wherein the cell targeting moiety is selected from the group consisting of an aptamer, antibody or part of an antibody or peptide.

Such pharmaceutical composition is disclosed, wherein the autoimmune diseases are selected from the group consisting of multiple sclerosis, type 1 diabetes, inflammatory bowel diseases, systemic lupus erythematosus, and rheumatic diseases.

The present disclosure relates to a method of preparing a pharmaceutical composition comprising the steps of:
culturing and collecting cells from an exosome depleted media;
isolating exosomes from the cell culture media by differential centrifugation followed by ultracentrifugation to form pellets of exosomes;
purifying the formed exosomes; and
encapsulating doubled stranded siRNA into the exosomes.

The present disclosure also relates to an inhibitor RNA molecule, wherein RNA is double standed and has having sequence GTGTTACTTTAGGACTTGGA and GCAGAAATCAGGGCGGAAAC.

The present disclosure further relates to a method of inhibiting a protein using an effective amount of the inhibitor RNA molecule.

Additionally, the present disclosure relates to a method for treating autoimmune diseases using the pharmaceutical composition, comprising administering an effective amount of the pharmaceutical composition.

1. A pharmaceutical composition for treating autoimmune diseases comprising:
a) a BLIMP1 beta inhibitor having a double stranded siRNA molecule with 10-21 nucleotides complementary to human PRDM1 gene mRNA for BLIMP1 beta protein;
(b) a pharmaceutically acceptable excipient; and
(c) a drug delivery vehicle comprising a nanocarrier,
wherein the double stranded SiRNA has a sequence GTGTTACTTTAGGACTTGGA and GCAGAAATCAGGGCGGAAAC.

2. The pharmaceutical composition as claimed in claim 1, further comprising a PEG.

3. The pharmaceutical composition as claimed in claim 1, wherein the double stranded siRNA molecule is encapsulated by exosomes.

4. The pharmaceutical composition as claimed in claim 1, further comprising a cell targeting moiety.

5. The pharmaceutical composition as claimed in claim 4, wherein the cell targeting moiety is selected from the group consisting of an aptamer, antibody or part of an antibody or peptide.

6. The pharmaceutical composition as claimed in claim 1, wherein the autoimmune diseases are selected the group consisting of multiple sclerosis, type 1 diabetes, inflammatory bowel diseases, systemic lupus erythematosus, and rheumatic diseases.

7. A method of preparing a pharmaceutical composition comprising the steps of:
culturing and collecting cells from an exosome depleted media;
isolating exosomes from the cell culture media by differential centrifugation followed by ultracentrifugation to form pellets of exosomes;
purifying the formed exosomes; and
encapsulating doubled stranded siRNA into the exosomes.

8. An inhibitor RNA molecule, wherein RNA is double standed and has having sequence GTGTTACTTTAGGACTTGGA and GCAGAAATCAGGGCGGAAAC.

9. A method of inhibiting a protein using an effective amount of the inhibitor RNA molecule as claimed in claim 8.

10. A method for treating autoimmune diseases using the pharmaceutical composition as claimed in claim 1, comprising administering an effective amount of the pharmaceutical composition.


SEQUENCE LISTING (2 sequences)

SEQ ID NO: 1 - RNA, 20 nt, Homo sapiens
  source 1..20: mol_type = other RNA
gtgttactttaggacttgga

SEQ ID NO: 2 - RNA, 20 nt, Homo sapiens
  source 1..20: mol_type = other RNA
gcagaaatcagggcggaaac